(19) United States
(12) Patent Application Publication
(10) Pub. No.: US 2025/0341490 A1
(43) Pub. Date: Nov. 6, 2025

(54) ELECTROLYTE FOR ELECTROCHEMICAL GAS SENSOR AND ELECTROCHEMICAL GAS SENSOR INCLUDING THE SAME

(71) Applicant: Samsung Electronics Co., Ltd., Suwon-si (KR)

(72) Inventors: Dongjoon Lee, Suwon-si (KR); Bokyung Jung, Suwon-si (KR)

(21) Appl. No.: 19/266,393

(22) Filed: Jul. 11, 2025

Related U.S. Application Data
(62) Division of application No. 17/846,211, filed on Jun. 22, 2022.

(30) Foreign Application Priority Data
Jul. 2, 2021 (KR) 10-2021-0087447

(51) Int. Cl. G01N 27/413; C07D 207/04; C07D 221/00; G01N 27/404

(52) U.S. Cl. CPC G01N 27/413; C07D 207/04; C07D 221/00; G01N 27/4045

(57) ABSTRACT

An electrolyte for an electrochemical gas sensor, the electrolyte including a protonic ionic liquid, wherein the protonic ionic liquid has an octanol-water partition coefficient LogP of about −3.5 or less, and wherein water is used as a reactant of an electrochemical reaction for gas sensing, the water is generated as a product of the electrochemical reaction for gas sensing, or a combination thereof.

### CROSS-REFERENCE TO RELATED APPLICATION

This application is divisional application of U.S. application Ser. No. 17/846,211, filed on Jun. 22, 2022, which is based on and claims priority to Korean Patent Application No. 10-2021-0087447, filed on Jul. 2, 2021, in the Korean Intellectual Property Office, and all benefits accruing therefrom under 35 U.S.C. § 119, the entire contents of which are incorporated by reference herein.

### BACKGROUND

### 1. Field

The present disclosure relates to electrolytes for electrochemical gas sensors and electrochemical gas sensors including the same.

### 2. Description of the Related Art

An electrochemical gas sensor is an electrochemical cell including two or more electrodes that are in contact with each other via an electrolyte. These electrochemical cells are typically open to the ambient atmosphere. A gas is introduced into one of the electrodes and the introduced gas is electrochemically converted and creates an electrical signal. A current generated, and thus the signal generated, by the conversion is proportional to the concentration of gas present or detected. For example, such a signal may be used to provide a hazardous gas detection alarm that is generated from the current. Sulfuric acid is one of the most common aqueous electrolytes used in sensors for detecting a gas such as CO, H₂S, or O₂.

An electrochemical gas sensor using an aqueous electrolyte such as sulfuric acid needs to include a water reservoir because the electrolyte is volatile or otherwise unavailable as a liquid in the non-aqueous state. However, such a water reservoir typically occupies more than 90% of the total sensor volume (e.g., sensor size or sensor footprint), which adds considerable size to the electrochemical gas sensor device, and thus miniaturization is challenging. As a way to solve this problem, various methods of using a non-volatile electrolyte instead of an aqueous electrolyte have been proposed.

Although a sensor may be miniaturized by using such non-volatile electrolytes, the commercially available non-volatile electrolytes known to date are ionic liquids that decompose at a high voltage, and thus a current fails to flow therein (e.g., the sensor fails at higher voltages). Thus, little sensitivity gain has been obtained by this method and there remains a need for improved electrolytes for use in electrochemical gas sensors.

### SUMMARY

Provided are non-volatile and hydrophilic electrolytes for electrochemical gas sensors.

Provided are electrochemical gas sensors miniaturizable and having improved sensitivity by including an electrolyte.

Provided are protonic ionic liquids and electrochemical devices including the same.

Additional aspects will be set forth in part in the detailed description that follows and, in part, will be apparent from the detailed description, or may be learned by practice of the presented one or more exemplary embodiments of the disclosure.

According to an aspect, an electrolyte for electrochemical gas sensors includes a protonic ionic liquid, wherein the protonic ionic liquid has an octanol-water partition coefficient LogP of about −3.5 or less, wherein water is used as a reactant of an electrochemical reaction for gas sensing, the water is generated as a product of the electrochemical reaction for gas sensing, or a combination thereof.

According to another aspect, an electrochemical gas sensor includes the above-described electrolyte for electrochemical gas sensors.

According to an embodiment, a protonic ionic liquid is represented by Formula 2.

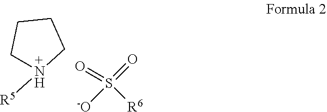

In Formula 2, R⁵ is a substituted or unsubstituted C2-C10 alkyl group, R⁶ is OH, —NH₂, or —NHR, and R is a substituted or unsubstituted C1-C10 alkyl group or a substituted or unsubstituted C6-C20 aryl group.

According to another aspect, an electrochemical device includes an electrolyte including the above-described protonic ionic liquid.

The electrochemical device is an electrochemical gas sensor, a battery, a biosensor, or a fuel cell.

### BRIEF DESCRIPTION OF THE DRAWINGS

The above and other aspects, features, and advantages of certain embodiments of the disclosure will be more apparent from the following detailed description taken in conjunction with the accompanying drawings, in which:

FIG. 1 is a schematic structure of an electrochemical gas sensor according to one or more embodiments;

FIG. 2A is a schematic structure of an electrochemical gas sensor according to one or more embodiments;

FIGS. 2B to 2F are schematic structures of electrochemical gas sensors according to one or more embodiments;

FIG. 3 is a graph of sensitivity (nanoamperes per part per million square centimeters, nA/ppm cm²) versus logP for protonic ionic liquids having various octanol-water partition coefficients;

FIG. 4 is a graph of current (microamperes, μA) versus voltage (Volts, V vs SHE) and shows current changes with respect to voltage by O₂ gas and CO gas in an protonic ionic liquid having a pKa of an anion's conjugate acid of −14, an protonic ionic liquid having a pKa of −3, and an protonic ionic liquid having a pKa of 5;

FIG. 5 is a graph of current (μA) versus voltage (V vs SHE) and shows current changes with respect to voltage by O₂ gas and CO gas in an protonic ionic liquid having a pKa of an anion's conjugate acid of −20;

FIG. 6A is a graph of is a graph of current (A) versus time (seconds, sec) illustrating current changes over time in an electrochemical gas sensor of Example 1; and

FIG. 6B is a graph of current (μA) versus time (sec) illustrating current changes over time in an electrochemical gas sensor of Comparative Example 17.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

The terminology used herein is for the purpose of describing one or more exemplary embodiments only and is not intended to be limiting. As used herein, the singular forms “a,” “an,” and “the” are intended to include the plural forms as well, unless the context clearly indicates otherwise. The term “or” means “and/or.” As used herein, the term “and/or” includes any and all combinations of one or more of the associated listed items. Expressions such as “at least one of,” when preceding a list of elements, modify the entire list of elements and do not modify the individual elements of the list. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

It will be understood that when an element is referred to as being “on” another element, it can be directly in contact with the other element or intervening elements may be present therebetween. In contrast, when an element is referred to as being “directly on” another element, there are no intervening elements present.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

Hereinafter, an electrolyte for electrochemical gas sensors and a gas sensor including the same according to an embodiment will be described in more further detail.

An electrolyte for electrochemical gas sensors includes a protonic ionic liquid, wherein the protonic ionic liquid has an octanol-water partition coefficient LogP of about −3.5 or less, and wherein water is used as a reactant of an electrochemical reaction for gas sensing, the water is generated as a product of the electrochemical reaction for gas sensing, or a combination thereof (e.g., wherein water participates in an electrochemical reaction for gas sensing in the electrochemical gas sensor).

Gas sensors may be classified into electrochemical, catalytic combustion, semiconductor, and optical gas sensors, according to the detection method. The present disclosure provides electrolytes used in electrochemical gas sensors, and among them, an electrolyte for electrochemical gas sensors in which water participates in an electrochemical reaction for gas sensing is provided.

Sulfuric acid is known as an aqueous electrolyte of electrochemical gas sensors. Although the aqueous electrolyte is sensitive to a target gas, an electrolyte reservoir, such as a water reservoir, is necessary because of the volatility of the aqueous electrolyte and the necessity for replenishment of the electrolyte in a high-temperature/high-humidity environment in the cases of using the aqueous electrolyte in a cell open to the ambient atmosphere. However, because the electrolyte reservoir can occupy 90% or of a total volume or size, it is practically difficult to miniaturize the electrochemical gas sensor. A method of using a non-volatile ionic liquid such as 1-ethyl-3-methylimidazolium bis(trifluoromethylsulfonyl)imide (EMI-TFI) instead of using the aqueous electrolyte has been proposed.

When the above-described non-volatile electrolyte is used, it is possible to minimize or reduce the size of the electrochemical sensor. The above-described non-volatile ionic liquid has high interface resistance, and thus a voltage difference applied to electrodes of the electrochemical gas sensor should be increased. However, the non-volatile ionic liquid may be decomposed at a higher voltage to cause an electrolyte decomposition current to flow in the electrochemical gas sensor, making it impossible to detect the target gas. As a result, it is difficult to perform gas sensing for the target gas.

The electrolyte for electrochemical gas sensors of one or more embodiments of the present disclosure are provided to solve the above-described and other problems. Because the electrolyte includes a non-volatile and hydrophilic protonic ionic liquid, the protonic ionic liquid, particularly, anions, are not decomposed at a higher temperatures, thereby providing excellent sensitivity to the target gas for sensing.

A system wherein water participates in an electrochemical reaction for gas sensing is applied to the electrochemical gas sensor. The system in which water participates in an electrochemical reaction for gas sensing includes both a case in which water is used as a reactant of the electrochemical reaction for gas sensing, a case in which water is generated as a product of the electrochemical reaction, or a combination thereof.

Because the protonic ionic liquid is hydrophilic, the protonic ionic liquid may help movement of water without blocking the approach of water although the protonic ionic liquid forms an interfacial alignment in an interface of electrodes. When the electrolyte including such a protonic ionic liquid is used, unlike the aqueous electrolyte, there is no need for a water reservoir, and thus it is possible to miniaturize and subminiaturize the electrochemical gas sensor and sensitivity to the target gas increases. Thus, such miniaturized and subminiaturized sensors may be used in mobile devices, internet of things (LoT), biosensors, and the like and may be used with a low power. In this regard, the biosensor is used to detect various harmful gases.

The electrolyte for electrochemical gas sensors includes a protonic ionic liquid, wherein the protonic ionic liquid has an octanol-water partition coefficient LogP of about −3.5 or less.

When the octanol-water partition coefficient LogP of the protonic ionic liquid is less than about −3.5, resistance decreases due to the protonic ionic liquid adsorbed to and aligned on the surface of the electrode, thereby minimizing the interface resistance of electrodes in the electrochemical gas sensor. When the octanol-water partition coefficient LogP of the protonic ionic liquid exceeds about −3.5, the interface resistance of the electrodes increases and thus a sensing voltage difference of the gas sensor, e.g., electrochemical gas sensor, using the protonic ionic liquid, should be increased resulting in a decrease in sensitivity.

The octanol-water partition coefficient of the protonic ionic liquid is, for example, from about −4.5 to about −3.5.

The octanol-water partition coefficient is a constant representing distribution of a solute in two immiscible phases, octanol and water. The octanol-water partition coefficient Kow may be expressed by LogP using Equation 1 below.

$$Log P = ( solubility of protonic ionic liquid in octanol ) \text{} / solubility of protonic ionic liquid in water ) \qquad Equation 1$$

When the electrolyte for electrochemical gas sensors includes the protonic ionic liquid having the above-described octanol-water partition coefficient, surface resistance may be stably maintained without a visible change under high-humidity or high-temperature/high-humidity conditions.

The protonic ionic liquid may include a protonic cation having a water binding energy of about 0.6 kilocalories per mole (Kcal/mol) or less, for example, about 0.001 to about 0.6 Kcal/mol. In addition, the protonic ionic liquid may include an anion having a water binding energy of about 7 Kcal/mol or less, for example, about 0.001 to 7 Kcal/mol. For example, within the ranges of the water binding energy, stability of the protonic ionic liquid may be easily obtained under high-humidity or high-temperature/high-humidity conditions.

The protonic ionic liquid for electrochemical gas sensors is not particularly limited as long as the protonic ionic liquid has an octanol-water partition coefficient within the range described above and, if necessary, a water binding energy within the range described above.

The protonic cation is represented by the following formula.

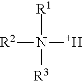

In the formula, R¹ to R³ are each independently hydrogen, a substituted or unsubstituted C1-C20 alkyl group, a substituted or unsubstituted C2-C20 alkoxyalkyl group, a substituted or unsubstituted C6-C20 aryl group, a substituted or unsubstituted C7-C20 alkylaryl group, or a substituted or unsubstituted C7-C20 arylalkyl group, and two or more of R¹ to R³ are linked to each other to form a ring. In this regard, the ring may be, for example, a substituted or unsubstituted pyrrolidine, a substituted or unsubstituted piperidine, or the like. A substituent of the substituted pyrrolidine and the substituted piperidine may be, for example, a halogen atom, a C1-C20 alkyl group substituted with a halogen atom (e.g., CCF₃, CHCF2, CH₂F, and CCl₃), a C1-C20 alkoxy group, a C2-C20 alkoxyalkyl group, a hydroxyl group, a nitro group, a cyano group, an amino group, an amidino group, hydrazine, hydrazone, a carboxylic acid group or a salt thereof, a sulfonyl group, a sulfamoyl group, a sulfonic acid group or a salt thereof, a phosphoric acid group or a salt thereof, a C1-C20 alkyl group, a C2-C20 alkenyl group, a C2-C20 alkynyl group, a C1-C20 heteroalkyl group, a C6-C20 aryl group, a C7-C20 arylalkyl group, C7-C20 alkylaryl group, C6-C20 aryloxy group, a C7-C20 aryloxyalkyl group, a C6-C20 heteroaryl group, a C2-C20 heteroarylalkyl group, C2-C20 alkylheteroaryl group, a C1-C20 heteroaryloxy group, or a C2-C20 heteroaryloxyalkyl group.

In the formula, R¹ to R³ may be a methyl group, an ethyl group, CH₃OCH₂—, CH₃CH₂OCH₂CH₂—, CH₃OCH₂CH₂— or two or more of R¹ to R³ may be linked to one another and nitrogen (N) to form a ring represented by one of Formula 1-1 below.

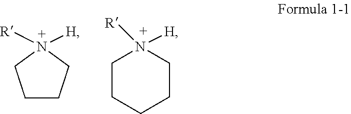

In Formula 1-1, R′ is a substituted or unsubstituted C1-C5 alkyl group.

In the Formula 1-1, the ring may be represented by one of Formula 1-2.

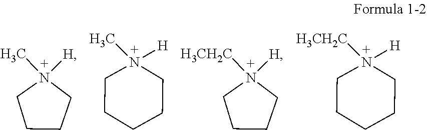

In the specification, the “octanol-water partition coefficient” may be obtained by using a commercial thermodynamics program, COSMOtherm (version C30_1301, COSMOlogic). By using the program, surface charge distribution of each compound and chemical potential difference to water and octanol were calculated to obtain the partition coefficient LogP, and GGA: BP86_TZP parameter is used in this process.

In the specification, the “binding energy” may be calculated using a density functional theory (DFT) calculation package, DMol3 (Materials Studio DMol3 version 6.1) manufactured by Biovia. BP functional/DNP basis was used as calculation levels. Optimized structures of a case where two materials are independently in gaseous phases and a case where the two materials are bound were obtained by modeling the two materials whose binding energies are to be measured in a single molecular level, and then a difference of energy between the two cases may be calculated as a binding energy.

In the protonic ionic liquid, a pKa of an anion's conjugate acid is about −14 or more, or from about −10 to about 7, or from about −3 to about 5. When the pKa of the anion's conjugate acid of the protonic ionic liquid is within the range described above, a decomposition voltage of the protonic ionic liquid increases, thereby effectively inhibiting decomposition of the protonic ionic liquid under high-voltage conditions.

The protonic ionic liquid-containing electrolyte having a vapor pressure of about 0.1 pascal (Pa) or less, for example, about 0.001 Pa to about 0.01 Pa, at 25° C. has non-volatility. The protonic ionic liquid-containing electrolyte may have an ionic conductivity of 10⁻⁷ S/cm or more, for example, about 1×10⁻⁷ Siemens per centimeter (S/cm) to about 1×10⁻² S/cm. When the electrolyte including the protonic ionic liquid having the above-described vapor pressure and ionic conductivity is used, a sensing current increases thereby increasing sensitivity of the electrochemical gas sensor.

The protonic ionic liquid has hydrophilicity higher than that of water and is immiscible with a compound having a dielectric constant lower than about 22 including at least one of acetone, isopropyl alcohol, ethyl acetate, dichloromethane, chloroform, tetrahydrofuran, diethylether, or a combination thereof.

A viscosity of the protonic ionic liquid is about 100 centipoise (cps) or less, about 90 cps or less, about 80 cps or less, for example, from about 5 cps to about 80 cps. When the protonic ionic liquid having the above-described viscosity is used, a sensing current increases thereby increasing sensitivity of the electrochemical gas sensor.

An electrochemical gas sensor according to another embodiment includes the above-described electrolyte for electrochemical gas sensors. The electrochemical gas sensor may measure a concentration of external gas by using a non-volatile and hydrophilic protonic ionic liquid.

In one or more embodiments, the electrochemical sensor may include two or more electrodes ionically in contact with the electrolyte including the protonic ionic liquid, and wherein the two or more electrodes are electrically insulated from each other by one or more separators or spaces. In addition, the electrochemical gas sensor may operate in various measurement methods, for example, by a method of measuring a current.

The electrochemical gas sensor may be used to detect and measure an acid gas, a neutral gas, an oxidizing gas, or a combination thereof.

The electrolyte for electrochemical gas sensors has a high solubility of a target gas. The target gas may include at least one, for example, CO, H₂S, SO₂, NO, NO₂, O₂, O₃, or a combination thereof.

When the target gas is oxygen, electrochemical reaction schemes may be different between a case in which water participates in the reaction as a reactant or product and a case in which water does not participates in the reaction. When water does not participate in the reaction, an oxygen anion radical generated as a product. The oxygen radical decomposes the electrolyte, thereby deteriorating performance of the sensor over the long term. To maintain long-term performance of the sensor, participation of water is essential to the electrochemical reaction.

An oxidation half-reaction potential of the protonic ionic liquid of the electrolyte is greater than an oxidation potential of at least one gas from CO, H₂S, SO₂, or NO, and a oxidation half-reaction potential of the protonic ionic liquid is from about 1.5 V to about 3 V. The oxidation half-reaction potential of the protonic ionic liquid is a voltage at which the protonic cation or anion of the protonic ionic liquid is oxidized to allow a decomposition reaction of the protonic ionic liquid to proceed. For example, oxidation potentials of CO, H₂S, SO₂, and NO are 0 V, 0 V, 0 V to 200 millivolts mV, and 300 mV, respectively.

The reduction half reaction potential of the protonic ionic liquid of the electrolyte is higher than reduction potentials of at least one of NO₂, O₂, and O₃ gases, and the decomposition voltage of the protonic ionic liquid is from −2 V to −1 V. The reduction half reaction potential of the protonic ionic liquid is a voltage at which the protonic cation or anion of the protonic ionic liquid is reduced to allow a decomposition reaction of the protonic ionic liquid to proceed. For example, reduction voltages of NO₂, O₂, and O₃ are from −200 mV to 0 V, −600 mV, and from −200 V to 0 V, respectively.

A usage time of the electrochemical gas sensor according to one or more embodiments may vary according to the use thereof and may be, for example, from about 1 week to about 5 years.

The electrochemical gas sensor according to one or more embodiments is applied to a system in which water participates in an electrochemical reaction for gas sensing. An exemplary electrochemical reaction occurring in the electrochemical gas sensor and potentials are as shown in Reaction Schemes 1 to 7 below. CO, H₂S, SO₂, NO, O₂, NO₂, and 03 are used as gases detected in Reaction Schemes 1 to 7, and water is provided as a reactant or product of the electrochemical reaction.

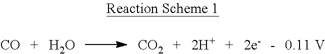

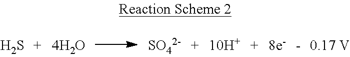

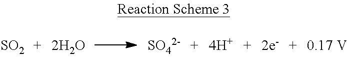

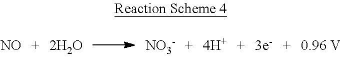

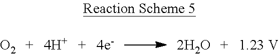

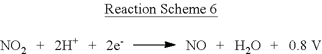

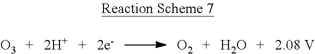

As shown in Reaction Schemes 1 to 7 above, water is used as a redox medium in the electrochemical gas sensor according to one or more embodiments.

The protonic ionic liquid for the electrochemical gas sensor according to one or more embodiments may be a compound represented by Formula 1 below.

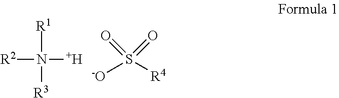

In Formula 1, R¹ to R³ are each independently hydrogen, a substituted or unsubstituted C1-C20 alkyl group, a substituted or unsubstituted C2-C20 alkoxyalkyl group, a substituted or unsubstituted C6-C20 aryl group, a substituted or unsubstituted C7-C20 alkylaryl group, or a substituted or unsubstituted C7-C20 arylalkyl group,
two or more of R¹ to R³ are linked to each other to form a ring,
R⁴ is OH, NH₂, NH (R⁵), or N (R⁵) (R⁶), and R⁵ and R⁶ are each independently a substituted or unsubstituted C1-C10 alkyl group, a substituted or unsubstituted C6-C20 aryl group, a substituted or unsubstituted C7-C20 alkylaryl group, or a substituted or unsubstituted C7-C20 arylalkyl group.

Throughout the specification, the C6-C20 aryl group may be, for example, a phenyl group or a substituted phenyl group.

In Formula 1, R¹ to R³ may be a methyl group, an ethyl group, CH₃OCH₂—, CH₃CH₂OCH₂CH₂—, or CH₃OCH₂CH₂— or two or more of R¹ to R³ may be linked to each other and nitrogen (N) to form a ring represented by one of Formula 1-1.

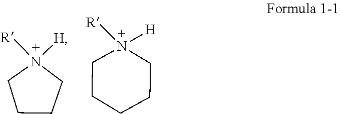

In Formula 1-1 R′ is a substituted or unsubstituted C1-C5 alkyl group. The ring may be represented by one of Formula 1-2.

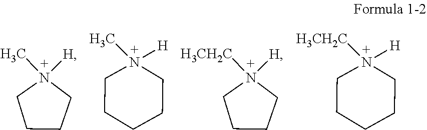

In Formula 1, R⁴ is, for example —NH₂, —NHCH₃ or —NHCH₂CH₃.

For example, the protonic ionic liquid for the electrochemical gas sensor according to one or more embodiments a compound represented by one of Formulae 6 to 22.

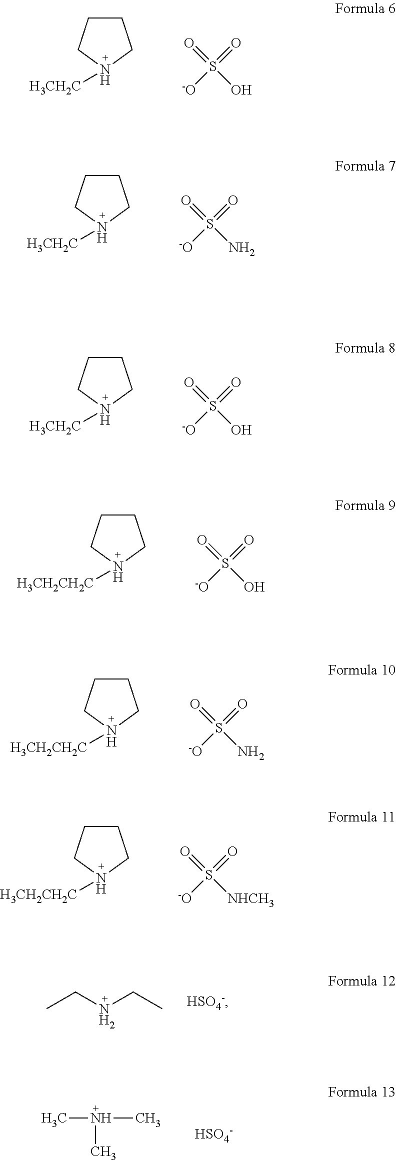

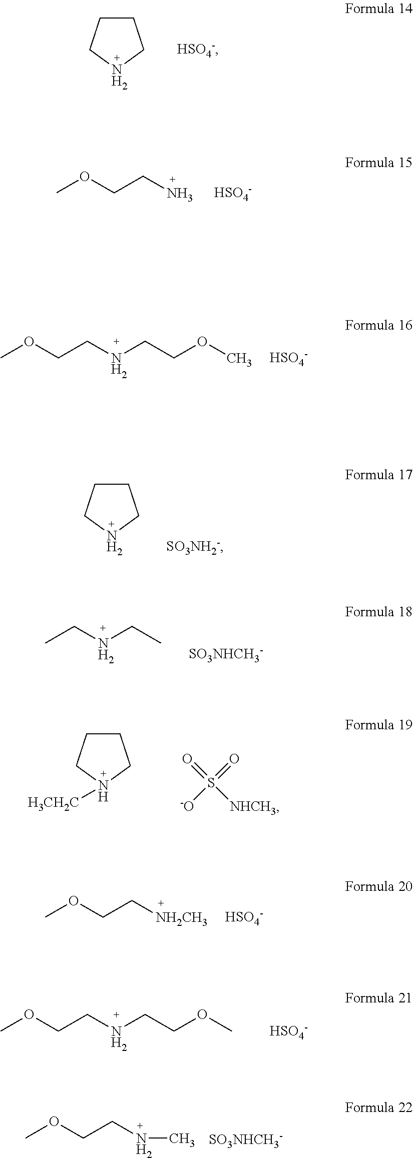

The electrolyte according to one or more embodiments may not contain water or may contain 1 wt % or less of water based on 100 wt % of the total weight of the electrolyte. Unlike conventional electrochemical gas sensors including an aqueous electrolyte, the electrochemical gas sensor according to one or more embodiments includes a very low amount of water and does not require an electrolyte reservoir, and thus a miniaturized and subminiaturized electrochemical gas sensor may be manufactured.

A content of the protonic ionic liquid in the electrolyte for electrochemical gas sensors may be from about 30 parts by weight to about 100 parts by weight, from about 70 parts by weight to about 100 parts by weight, or from about 99 parts by weight to about 100 parts by weight. When the content of the protonic ionic liquid is within the ranges described above, an electrochemical gas sensor having excellent sensitivity and increased usable time may be manufactured.

In the electrolyte for electrochemical gas sensors according to one or more embodiments, a water content may be about 70 parts by weight or less, from about 0.001 parts by weight to about 70 parts by weight, from about 0.001 parts by weight to about 30 parts by weight, or from about 0.001 parts by weight to about 1 part by weight based on 100 parts by weight of the total weight of the electrolyte, and

an amount of water evaporation after 5 minutes may be about 0.01 wt % or less when the ambient temperature of the electrochemical gas sensor is 25° C.

The electrochemical gas sensor according to one or more embodiments may undergo an aging process at room temperature (25° C.). Through the aging process, equilibrium with the atmospheric environment is controlled, resulting in improvement sensitivity for gas sensing. An aging time may vary according to size, dimension, sensing gas, and the like of the electrochemical gas sensor, and may be for example, from about 1 day to about 5 days or from about 1 day to about 3 days.

The electrolyte for electrochemical gas sensors may further include at least one additive that may be an organic additive, an organometallic additive, an inorganic additive, or a combination thereof. In this regard, a content of the additive may be from about 0.001 wt % to about 15 wt % based on the total weight of the electrolyte.

In the electrochemical gas sensor, a target gas is detected by electrochemical reaction among gases introduced into the sensor, the two or more electrodes, and the electrolyte of the sensor.

In one or more embodiments, the electrochemical gas sensor includes two or more electrodes in contact with the electrolyte including the protonic ionic liquid, wherein the two or more electrodes are electrically separated from each other (e.g., by separators or spaces). Two-electrode, three-electrode, and multiple-electrode sensor systems may be formed. In various representative embodiments, a two-electrode or three-electrode system is formed.

The two-electrode system includes one working electrode WE (first electrode) and a counter electrode CE (second electrode). In the case of the three-electrode system, a reference electrode RE (third electrode) is further used. In the multiple-electrode system, the electrochemical gas sensor may further include a protective electrode or more than one working electrode.

According to one or more embodiments, oxidation or reduction of an introduced gas occurs in the working electrode, ions diffused toward the counter electrode are formed by the electrolyte, and deprotonation may occur in the counter electrode. Such reactions result in a flow of a current detectable by a galvanic cell. Therefore, the flow of the current indicates presence of the gas (reactive species) to be detected.

The electrodes may each independently include at least one metal from Cu, Ni, Ti, Pt, Ir, Au, Pd, Ag, Ru, or Rh, an oxide of at least one metal from Cu, Ni, Ti, Pt, Ir, Au, Pd, Ag, Ru, or Rh, a mixture thereof, or carbon.

The electrochemical gas sensor according to one or more embodiments may operate at a high voltage of 1 V or greater.

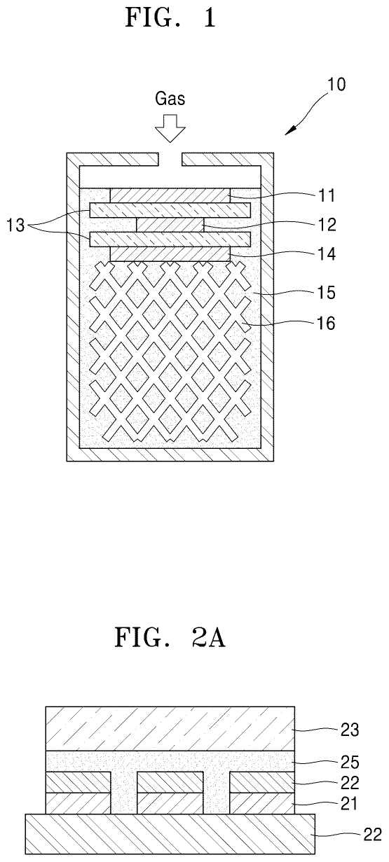

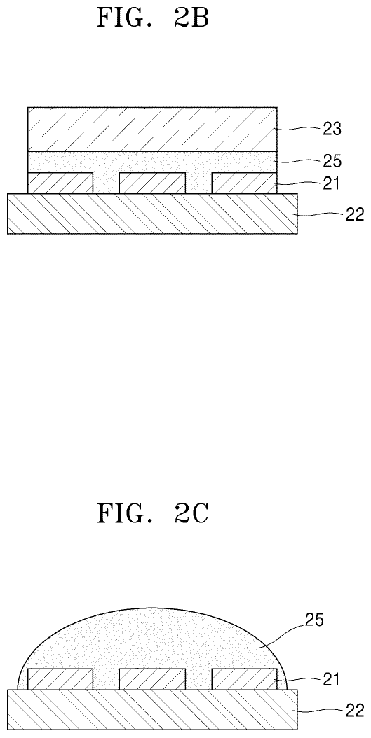

FIGS. 1, and 2A to 2C are schematic diagrams illustrating electrochemical gas sensors according to one or more embodiments. The electrochemical gas sensor may be, for example, a CO sensor. Among them, FIG. 1 illustrates a structure of a miniaturized electrochemical gas sensor, and FIGS. 2A to 2C illustrate structures of subminiaturized electrochemical gas sensors.

Referring to FIG. 1, an electrochemical sensor 10 includes three electrodes, i.e., a working electrode 11, a counter electrode 14, and a reference electrode 12, which are ionically in contact with an electrolyte 15 including a protonic ionic liquid and electrically insulated from each other by one or more separators 13. The counter electrode 14 and the reference electrode 12 are installed to face the working electrode 11 exposed to a CO gas atmosphere. Oxidation (or reduction) reaction occurs at the working electrode, and simultaneously, reduction (or oxidation) reaction occurs at the counter electrode, and then the reference electrode detects a potential changing along with the oxidation and reduction reactions and maintains the potential at a constant level.

The electrochemical gas sensor 10 includes a gas inlet and at least one gas outlet, a housing 17 filled with the electrolyte 15 according to one or more embodiments, the working electrode 11, the counter electrode 14, and the reference electrode 12 as shown in FIG. 1. The separators 13 are interposed between the working electrode 11 and the reference electrode 12 and between the reference electrode 12 and the counter electrode 14, and a moisturizing member 16 is provided to contain water. As the moisturizing member 16, cotton wool may be used.

Currents flowing between the working electrode 11 and the reference electrode 12 and between the reference electrode 12 and the counter electrode 14 are measured to determine a concentration of CO gas.

The working electrode 11 is a gas diffusion electrode in the housing. The working electrode 11 includes, for example, Pt-C, and the counter electrode includes, for example, Pt-C.

The working electrode, the counter electrode, and the reference electrode may include, for example, at least one metal from platinum (Pt), gold (Au), palladium (Pd), ruthenium (Ru), nickel (Ni), stainless steel (STS), aluminum (AI), molybdenum (Mo), chromium (Cr), copper (Cu), titanium (Ti), tungsten (W), tin-doped In₂O₃ (ITO), fluorine-doped SnO₂ (FTO), iridium (Ir), silver (Au), or rhodium (Rh), an oxide of the at least one metal, a mixture thereof, carbon, or a combination thereof. Materials for the electrodes may be the same or different. The metal oxide may include at least one, for example, SnO₂, TiO₂, ZnO, VO₂, In₂O₃, NiO, MoO₃, SrTiOs, Fe-doped SrTiO₃, Fe₂O₃, WO₃, or CuO.

The electrode may have any suitable shape. In several representative studies, potentials of working electrodes were generally maintained at a constant level. However, the potential of the working electrode may vary.

The separator 13 may be formed of glass fibers or a silicate structure saturated with a liquid electrolyte.

The electrochemical gas sensor is connected to an electronic measurement system that maintains a potential difference between the working electrode and the reference electrode and amplifies a sensor current to provide a measured signal.

When the working electrode is exposed to a carbon monoxide atmosphere, a reaction as shown in Reaction Scheme 8 occurs and a reaction as shown in Reaction Scheme 9 occurs in the counter electrode as water is supplied to the working electrode.

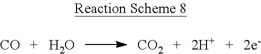

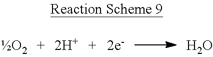

Referring to FIG. 2A, a plurality of electrodes 21 are arranged on a substrate 22, a catalyst material-containing layer 24 on the electrodes 21, and an electrolyte 25 for electrochemical gas sensors according to one or more embodiments is provided between the catalyst material-containing layer 24l and a porous gas-permeable membrane 23. The catalyst material-containing layer 24 includes, for example, Pt/C. The substrate 22 is formed of a glass, alumina, silicon, or silicon oxide substrate, and the electrode 21 is a Pt electrode.

Through the porous gas-permeable membrane 23, the sensor may communicate with the ambient atmosphere in a fluid communication manner, and the porous gas-permeable membrane 23 may be, for example, a porous polytetrafluoroethylene membrane.

An electrochemical gas sensor of FIG. 2B has the same structure as that of the electrochemical gas sensor of FIG. 2A, except for not including the catalyst material-containing layer 24. In addition, an electrochemical gas sensor of FIG. 2C is provided such that an electrolyte 25 is in the form of droplets.

The electrochemical gas sensor of FIG. 2A is a miniaturized sensor in which a diameter (X-axial or Y-axial length) of the electrolyte is from about 0.01 mm to about 3 mm, from about 0.1 mm to about 2 mm, or for example, about 1 mm, and a content of the electrolyte contained in the sensor is from about 0.1 microliter (μL) to about 10 μL, for example, about 1 μL. In addition, the electrochemical gas sensors of FIGS. 2B and 2C are subminiaturized sensors in which a diameter of the electrolyte is 2 millimeters (mm) or less, for example, about 1 mm or less, and a content of the electrolyte contained in the sensor is from about 2 μL to about 10 μL, when compared to that of FIG. 2A.

The electrolyte according to one or more embodiments may include an additive including an organic additive, an organometallic additive, and an inorganic additive. The additive may improve performance of the electrochemical gas sensor in terms of sensitivity, response time, selectivity, and robustness. The additive may be mixed with a protonic ionic liquid electrolyte. A content of the additive may be from about 0.05 wt % to about 15 wt %, from about 0.05 wt % to about 5.0 wt %, or from about 0.05 wt % to about 1.5 wt % based on 100 parts by weight of the total weight of the electrolyte.

The organic additive may include at least one of imidazole, pyridine, pyrrole, pyrazole, pyrimidine, guanine, uric acid, benzoic acid, porphyrin, a porphyrin derivative, substituted guanine, substituted uric acid, substituted benzoic acid, substituted porphyrin, substituted porphyrin derivative, ortho-quinone, para-quinone, substituted ortho-quinone, and substituted para-quinone, dihydroxynaphthalene, substituted dihydroxynaphthalene, anthraquinone, or substituted anthraquinone. Here, the term “substitution” indicates that at least one hydrogen in the functional group is substituted with a C1-C4 alkyl group. When the organic additive is added, a reference potential and/or H may be stabilized.

The organic additive may include water, propylene carbonate, ethylene carbonate, or a mixture thereof.

The organometallic additive may be a metal phthalocyanine including Mn²⁺, Cu²³⁰, Fe^{2+/3+}, or Pb²⁺ as a metal cation, or derivatives thereof. When the metal phthalocyanine is added, sensitivity to a particular gas, e.g., CO gas, may be substantially increased.

The inorganic additive may include at least one of an alkali halide, an ammonium halide, an ammonium halide substituted with at least one C1-C4 alkyl group, or a transition metal salt of Mn²⁺, Mn³⁺, Cu²⁺, Ag⁺, Cr³⁺, Cr⁶⁺, Fe²⁺, Fe³⁺, or Pb²⁺, for example lithium bromide, lithium iodide, ammonium iodide, tetramethylammonium iodide, tetraethylammonium iodide, tetrapropylammonium iodide, tetrabutylammonium iodide, tetrabutylammonium bromide, manganese (II) chloride, manganese (II) sulfate, manganese (II) nitride, chromium (III) chloride, alkali chromate, iron (II) chloride, iron (III) chloride, or lead (II) nitrate, e.g., an alkali halide such as Lil or NR₄l (where R is H, a methyl group, an ethyl group, a butyl group, or a mixture thereof) and/or an ammonium halide at a low percentage (e.g., from about 0.05% to about 15%). When such additives are added, sensitivity of the sensor to halogen gas and vapor may be increased.

The electrolyte may further include a conductive salt. The conductive salt may be, for example, 1-hexyl-3-methyl-imidazolium-tris(pentafluoroethyl)-trifluorophosphate.

FIG. 2D is a schematic diagram illustrating a structure of an electrochemical gas sensor including three electrodes according to another embodiment.

An electrochemical gas sensor 1 includes a working electrode 3, a reference electrode 5, and a counter electrode 6 in a sensor housing 2, wherein the working electrode 3 is located to communicate with the ambient atmosphere in a fluid communication manner through a gas-permeable membrane. The electrodes are physically separated from one another but ionically interconnected via a separator 4. The electrolyte according to one or more embodiments may be provided in an inner space of the sensor.

The separator may be formed of glass fibers or a silicate structure saturated with the electrolyte according to one or more embodiments. In addition, a supplementary volume 7 provides a volume for absorbing water in the case of an absorbent electrolyte. The sensor is connected to, for example, an electronic measurement system 8 capable of amplifying a sensor current to provide a measured signal.

FIG. 2E shows another electrochemical gas sensor 11 including a sensor housing 12. A working electrode 13a, a reference electrode 15, and a counter electrode 16 are arranged in a housing 12, and the working electrode 13a communicates with the ambient atmosphere in a fluid communication manner through a gas-permeable membrane 13. The electrodes are physically separated from one another but ionically interconnected via a separator 14. The electrolyte according to one or more embodiments may be provided in an inner space of the sensor.

The separator 14 may be formed of glass fibers or a silicate structure saturated with the electrolyte.

The reference electrode 15 and the counter electrode 16 are located in a line on the side of the separator 14 opposite to the working electrode 13a. The supplementary volume 17 provides a volume to which water is absorbed when atmospheric humidity changes. The electrochemical gas sensor 11 is connected to an electronic measurement system 18 that maintains a potential difference between the working electrode 13a and the reference electrode 15 and amplifies a sensor current to provide a measured signal.

FIG. 2F shows another embodiment of the electrochemical gas sensor 11 including the sensor housing 12. Referring to this, a working electrode 13a, a reference electrode 15, and a counter electrode 16 are arranged in a housing 12, and the working electrode 13a communicates with the ambient atmosphere in a fluid communication manner through a gas-permeable membrane 13. The working electrode 13a and the reference electrode 15 are physically separated from each other but ionically interconnected via a first separator 14a. A second separator 14b is interposed between the reference electrode 15 and the counter electrode 16. The first and second separators 14a and 14b may include the electrolyte according to one or more embodiments. The electrolyte according to one or more embodiments may be provided in an inner space of the sensor.

The above-described gas sensors of FIGS. 2D to 2F may be used to detect a nitrogen-containing compound, for example, may be used as an NH₃ sensor.

The electrochemical gas sensor according to one or more embodiments may be applied to mobile phones and wearable devices. Also, the sensors may be used as subminiaturized sensors to be mounted in mobile devices and the subminiaturized sensors may be used as portable health care biosensors for harmful gas warning and exhalation analysis.

Hereinafter, a protonic ionic liquid and an electrochemical gas sensor including an electrolyte including the same will be described in more detail.

According to another embodiment, a protonic ionic liquid is represented by Formula 2 below.

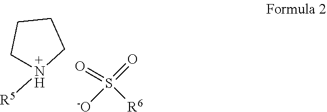

In Formula 2, R⁵ is a C2-C10 alkyl group,

R⁶ is OH, —NH₂, or —NHR, and R is a C1-C10 alkyl group, or a C6-C20 aryl group.

R is a C1 to C5 alkyl group or a C1 to C3 alkyl group. R is, for example, a methyl group, an ethyl group, or a propyl group.

R⁵ is an ethyl group, a propyl group, a butyl group, or a hexyl group.

The protonic ionic liquid of Formula 2 is a hydrophilic material and has nonvolatility due to a low vapor pressure. In addition, the protonic ionic liquid is in a liquid state unlike the protonic ionic liquid wherein R⁵ is a methyl group in Formula 2. Therefore, by using the protonic ionic liquid of Formula 2, an electrochemical gas sensor having nonvolatility and excellent sensitivity and requiring a liquid electrolyte may be manufactured. The protonic ionic liquid in which R⁵ is a methyl group in Formula 2 is in a solid state and thus it cannot be applied to the electrochemical gas sensor according to one or more embodiments.

The protonic ionic liquid of Formula 2 may a compound represented by one of Formulae 3 to 5.

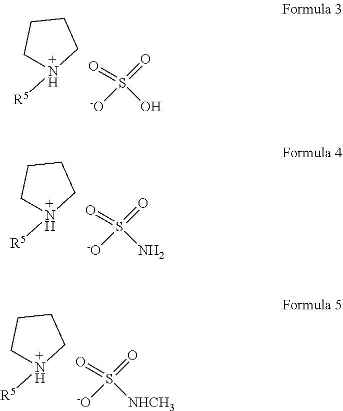

In Formulae 3 to 5, R⁵ is a substituted C2-C10 alkyl group.

R⁵ is an ethyl group, a propyl group, a butyl group, a pentyl group, or a hexyl group.

The above-described ionic liquid is a compound represented by one of Formulae 6 to 11.

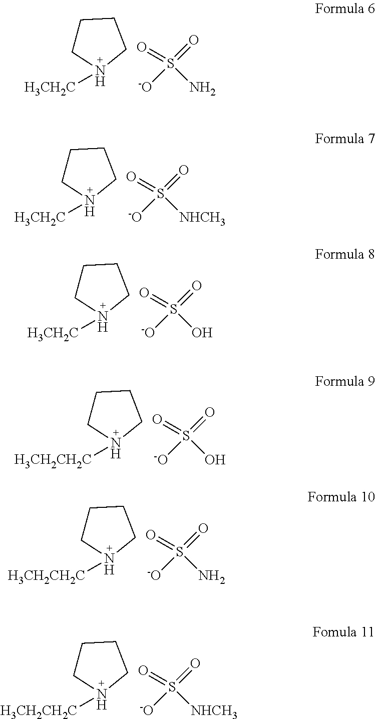

According to another embodiment, an electrochemical device includes the above-described the protonic ionic liquid-containing electrolyte.

The electrochemical device may be applied to, for example, gas sensors, fuel cells, biosensors, and plating solutions in the electrochemical filed. The gas sensor is, for example, an electrochemical gas sensor, and presence of a target gas may be detected by a change in electrical resistance or electrical conductivity.

The term “C1-C20 alkyl group” as used herein refers to a linear or branched saturated aliphatic hydrocarbon monovalent group having 1 to 20 carbon atoms, and examples thereof are a methyl group, an ethyl group, a propyl group, an isobutyl group, a sec-butyl group, a tert-butyl group, a pentyl group, an isoamyl group, a hexyl group, and the like.

The term “C2-C20 alkoxyalkyl group” as used herein refers to a C1-C20 alkyl group substituted with a monovalent group represented by -OA₁₀₁ (wherein Atot is a C1-C20 alkyl group).

The term “C2-C20 alkenyl group” as used herein refers to a hydrocarbon group formed by substituting at least one carbon-carbon double bond in the middle or at the terminus of the C2-C20 alkyl group, and examples thereof are an ethenyl group, a propenyl group, a butenyl group, and the like.

The term “C2-C20 alkynyl group” as used herein refers to a hydrocarbon group formed by substituting at least one carbon-carbon triple bond in the middle or at the terminus of the C2-C20 alkyl group, and examples thereof are an ethynyl group, a propynyl group, and the like.

The term “C6-C20 aryl group” as used herein refers to a monovalent group having a carbocyclic aromatic system having 6 to 20 carbon atoms. Examples of the C6-C20 aryl group are a phenyl group, a naphthyl group, and the like. When the C6-C20 aryl group includes two or more rings, the two or more rings may be fused to each other.

The term “C1-C20 heteroaryl group” as used herein refers to a monovalent group having a carbocyclic aromatic system that has at least one heteroatom selected from N, O, P, Si, Ge, Se, and S as a ring-forming atom, and 1 to 20 carbon atoms. Examples of the C1-C20 heteroaryl group include a pyridinyl group, a pyrimidinyl group, a pyrazinyl group, a pyridazinyl group, a triazinyl group, a quinolinyl group, an isoquinolinyl group, and the like. When the C2-C60 heteroaryl group includes two or more rings, the two or more rings may be fused to each other.

The term “C6-C20 aryloxy group” as used herein indicates -OA₁₀₂ (wherein A₁₀₂ is the C6-C20 aryl group).

The term “C1-C20 heteroaryloxy group” as used herein indicates -OA_{102′} (wherein A_{102′} is the C1-C20 heteroaryl group).

The term “C7-C20 alkylaryl group” as used herein refers to a C1-C20 aryl group substituted with at least one C1-C20 alkyl group. The term “C7-C20 arylalkyl group” as used herein refers to a C1-C20 alkyl group substituted with at least one C6-C20 aryl group.

The term “C2-C20 alkylheteroaryl group” as used herein refers to a C1-C20 heteroaryl group substituted with at least one C1-C20 alkyl group. The term “C2-C20 heteroarylalkyl group” as used herein refers to a C1-C20 alkyl group substituted with at least one C1-C20 heteroaryl group.

Throughout the specification, unless otherwise stated, the terms “substitution” and “substituted” indicate that at least one hydrogen in the functional group of the present disclosure is substituted with at least one substituent that may be a halogen atom (—F, —Cl, —Br or —I), a hydroxyl group, a nitro group, a cyano group, an amino group (NH₂, NH (R¹⁰⁰), or N (R¹⁰¹) (R¹⁰²), wherein R¹⁰⁰, R¹⁰¹, and R¹⁰² are identical or different and are each independently a substituted or unsubstituted C1 to C10 alkyl group or a substituted or unsubstituted C6-C20 aryl group), an amidino group, a hydrazine group, a hydrazone group, a carboxylic acid group or a salt thereof, a sulfonic acid group or a salt thereof, a phosphoric acid group or a salt thereof, a substituted or unsubstituted C1-C20 alkyl group, a substituted or unsubstituted C2-C20 alkenyl group, a substituted or unsubstituted C2-C20 alkynyl group, a substituted or unsubstituted C1-C20 alkoxy group, a substituted or unsubstituted C6-C20 aryl group, a substituted or unsubstituted C7-C20 alkylaryl group, a substituted or unsubstituted C7-C20 arylalkyl group, a substituted or unsubstituted C6-C20 aryloxy group, a substituted or unsubstituted C1-C20 heteroaryl group, a substituted or unsubstituted C2-C20 alkylheteroaryl group, a substituted or unsubstituted C2-C20 heteroarylalkyl group, or a substituted or unsubstituted C1-C20 heteroaryloxy group.

Hereinafter, the present disclosure will be described in more detail with reference to the following examples. However, the following experimental examples are merely presented to exemplify the present disclosure, and the scope of the present disclosure is not limited thereto.

### EXAMPLES

### Preparation of Protonic ionic liquid

### Preparation Example 1

Sulfuric acid (10.3 millimoles (mmol)) was added to 1 gram (g) of water, and diethylamine (10.3 mmol) was added dropwise thereto while stirring in an ice bath for 30 minutes, followed by evaporation under reduced pressure in an evaporator to obtain a protonic ionic liquid shown in Table 1 below.

### Preparation Example 2

A protonic ionic liquid shown in Table 1 below was obtained in a similar manner as in Preparation Example 1, except that trimethylamine was used instead of diethylamine.

### Preparation Example 3

An protonic ionic liquid shown in Table 1 below was obtained in a similar manner as in Preparation Example 1, except that pyrrolidine was used instead of diethylamine.

### Preparation Example 4

An protonic ionic liquid shown in Table 1 below was obtained in a similar manner as in Preparation Example 1, except that 1-ethylpyrrolidine was used instead of diethylamine.

### Preparation Example 5

An protonic ionic liquid shown in Table 1 below was obtained in a similar manner as in Preparation Example 1, except that 2-methoxyethylamine was used instead of diethylamine.

### Preparation Example 6

An protonic ionic liquid shown in Table 1 below was obtained in a similar manner as in Preparation Example 1, except that N-(2-methoxyethyl) methylamine was used instead of diethylamine.

### Preparation Example 7

An protonic ionic liquid shown in Table 1 below was obtained in a similar manner as in Preparation Example 1, except that bis(2-methoxyethyl) amine was used instead of diethylamine.

### Preparation Example 8

An protonic ionic liquid shown in Table 1 below was obtained in a similar manner as in Preparation Example 1, except that sulfamic acid was used instead of sulfuric acid and 1-ethylpyrrolidine was used instead of diethylamine.

### Preparation Example 9

An protonic ionic liquid shown in Table 1 below was obtained in a similar manner as in Preparation Example 1, except that methylsulfamic acid was used instead of sulfuric acid.

### Preparation Example 10

An protonic ionic liquid shown in Table 1 below was obtained in a similar manner as in Preparation Example 9, except that 1-ethylpyrrolidine was used instead of diethylamine.

### Preparation Example 11

An protonic ionic liquid shown in Table 1 below was obtained in a similar manner as in Preparation Example 9, except that 2-methoxyethylamine was used instead of diethylamine.

### Preparation Example 12

An protonic ionic liquidin Table 1 below was obtained in a similar manner as in Preparation Example 9, except that N-(2-methoxyethyl) methylamine was used instead of diethylamine.

Table 2 below shows protonic ionic liquids according to Comparative Preparation Examples 1 to 16, commercially available from Aldrich or TMI.

TABLE 1

| | Protonic ionic liquid | | |
|---|---|---|---|
| Example | Cation | Anion | Formula |
| 1 | diEtNH₂ | HSO₄⁻ | 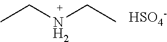 |
| 2 | triMeNH | HSO₄⁻ | 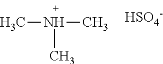 |
| 3 | PyrH | HSO₄⁻ | 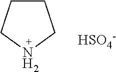 |
| 4 | EtPyrH | HSO₄⁻ | 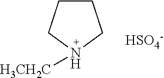 |
| 5 | MeOEtNH₃ | HSO₄⁻ | 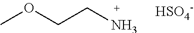 |
| 6 | MeOEtNH₂Me | HSO₄⁻ | 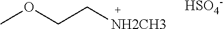 |
| 7 | bis(MeOEt)NH₂ | HSO₄⁻ | 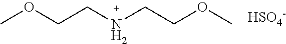 |
| 8 | EtPyrH | SO₃NH₂⁻ | 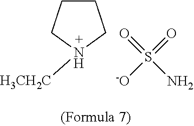 |
| 9 | diEtNH₂ | SO₃NHMe | 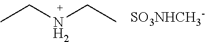 |
| 10 | EtPyrH | SO₃NHMe | 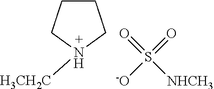 |
| 11 | MeOEtNH₃ | SO₃NHMe | 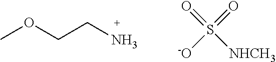 |
| 12 | MeOEtNH₂Me | SO₃NHMe | 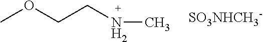 |

TABLE 2

| Comparative | | | |
|---|---|---|---|
| Preparation | Protonic ionic liquid | | |
| Example | Compound (abbreviation of compound) | Cation | Anion |
| 1 | 1-Ethyl-3-methylimidazolium | EMI | TFSI |
| | bis(trifluoromethanesulfonyl)imide (EMI-TFSI) | | |
| 2 | 1-Ethyl-3-methylimidazolium ethyl sulfate (EMI-EtSO4) | EMI | EtSO₄ |
| 3 | 1-Propyl-1-methylpyrrolidinium bis(trifluoromethanesulfonyl)imide | Pyr13 | TFSI |
| | (Pyr13-TFSI) | | |
| 4 | 1 -Butyl-1 -methylpyrrolidinium trifluoromethanesulfonate | Pyr14 | Otf |
| | (Pyr14-OTf) | | |
| 5 | 1-Butyl-1-methylpyrrolidinium dicyanamide (Pyr14-DCN) | Pyr14 | DCN |
| 6 | 1-Ethyl-3-methylimidazolium dicyanamide (EMI-DCN) | EMI | DCN |
| 7 | 1-Ethyl-3-methylimidazolium tricyanomethanide (EMI-CCN) | EMI | CCN |
| 8 | 1-Ethyl-3-methylimidazolium thiocyanate (EMI-SCN) | EMI | SCN |
| 9 | 1 -Ethyl-3-methylimidazolium trifluoroacetate (EMI-TFA) | EMI | TFA |
| 10 | 1-Ethyl-3-methylimidazolium Diethyl Phosphate(EMI-diEtPO₄) | EMI | diEtPO₄ |
| 11 | Diethylmethylammonium triflate (DEMHA-Otf) | DEMHA | Otf |
| 12 | 1-Butyl-3-methylimidazolium hydrogen sulfate (BMI-HSO₄) | BMI | HSO₄_{^{−}} |
| 13 | 1-Ethylimidazolium bis(trifluoromethylsulfonyl)imide (HEI-TFSI) | HEI | TFSI |
| 14 | 1-Ethylimidazolium nitrate (HEI-NO₃) | HEI | NO₃ |
| 15 | Ethylammonium nitrate (EH3A-NO₃) | EH3A | NO₃ |
| 16 | Propylammonium nitrate (PH3A-NO₃) | PH3A | NO₃ |

### Preparation of Electrochemical Gas Sensor

### Example 1

A cylindrical electrochemical gas sensor as shown in FIG. 1 was assembled using the protonic ionic liquid prepared in Preparation Example 1 as an electrolyte by using a CO Sensor Make Kit that is a small CO sensor making kit manufactured by SENKO (Korea). A content of the protonic ionic liquid in the electrolyte was 100 parts by weight based on 100 parts by weight of the total weight of the electrolyte. A diameter of the cylindrical electrochemical gas sensor was about 1.6 centimeters (cm) and the amount of the electrolyte was about 1 mL.

The electrochemical gas sensor was aged for about 3 days at room temperature (25° C.). Through this process, 30 parts by weight of water was included therein.

### Examples 2-12

Electrochemical gas sensors were prepared in a similar manner as in Example 1, except that the protonic ionic liquids prepared in Preparation Examples 2 to 12 were respectively used instead of the protonic ionic liquid of Preparation Example 1.

### Example 13

ED-SE1-Pt, which is a miniaturized three-electrode device manufactured by Micrux, was used as a substrate on which a plurality of electrodes of FIG. 2A are arranged.

Separately, a Pt/carbon catalyst (TEC10E50E, where carbon is Ketjen black, and a Pt content: 46.7 wt %, Tanaka Kikinzoku Kogyo K.K., Japan) and NAFION (Du Pont) were dispersed in isopropyl alcohol in a weight ratio of 1:0.2 to prepare a composition for a catalyst material-containing layer.

The composition for a catalyst material-containing layer was coated on the plurality of electrodes and dried to form a catalyst material-containing layer on Pt electrodes. Then, an electrolyte including the protonic ionic liquid of Preparation Example 1 was applied onto the catalyst material-containing layer. In the electrolyte, the content of the protonic ionic liquid was 100 parts by weight based on 100 parts by weight of the total weight of the electrolyte.

Then, a porous polytetrafluoroethylene membrane was located on the resultant to prepare an electrochemical gas sensor having a structure as shown in FIG. 2A. A diameter of the electrochemical gas sensor was about 1 mm, and an amount of the electrolyte was about 2 ul.

The electrochemical gas sensor was aged for about one day at room temperature. Through this process, 30 parts by weight of water was included therein based on 100 parts by weight of the electrolyte.

### Comparative Examples 1 to 16

Electrochemical gas sensors were prepared in a similar manner as in Example 1, except that the protonic ionic liquids prepared in Comparative Preparation Examples 1 to 16 were used instead of the protonic ionic liquid of Preparation Example 1.

### Comparative Example 17

An electrochemical gas sensor was prepared in a similar manner as in Example 13, except that 1 M H₂SO₄ was used as an electrolyte.

Evaluation Example 1: Nuclear Magnetic Resonance Spectrum and Infrared Spectroscopy Spectrum

Nuclear magnetic resonance (NMR) spectra of the protonic ionic liquids prepared according to Preparation Examples 4, 8, and 10 were obtained.

Proton (¹H) NMR and carbon (13C) NMR spectra analysis results of the compounds of Preparation Examples 4, 8, and 10 are as shown below.

Compound of Preparation Example 4: 1H NMR (500 megahertz (MHZ), dimethylsulfoxide-d6 (DMSO-d6) delta (δ, parts per million (ppm)) 1.20 (t, 3H), 1.82˜1.84(m, 2H), 1.95˜1.98 (m, 2H), 2.94˜2.98 (m, 2H), 3.11˜3.15 (m, 2H), 3.46˜3.49 (m, 2H), 9.46 (brs, 2H).

Compound of Preparation Example 4: 13C NMR (125 MHZ, DMSO-d6) δ 11.21, 22.99, 49.49, 53.11.

Compound of Preparation Example 8: 1H NMR (500 MHZ, DMSO-d6) δ 1.20 (t, 3H), 1.90˜1.92 (m, 4H), 3.10˜3.29 (m, 6H), 6.33 (br,s, 2H), 9.44 (br, s, 1H).

Compound of Preparation Example 8: 13C NMR (125 MHZ, DMSO-d6) δ 11.22, 23.04, 49.58, 53.13.

Compound of Preparation Example 10: 1H NMR (500 MHz, DMSO-d6) δ 1.20 (t, 3H), 1.90˜1.92 (m, 4H), 2.41 (s, 3H), 2.97˜3.37 (m, 6H), 7.06 (brs, 1H), 9.49 (br, s, 1H)

Compound of Preparation Example 10: 13C NMR (125 MHZ, DMSO-d6) δ 11.22, 23.03, 30.21, 49.55, 53.13.

Based on the results of the NMR analyses, structures of the protonic ionic liquid prepared in Preparation Examples 4, 8, and 10 were identified.

### Evaluation Example 2: Octanol-water Partition Coefficient (LogP)

Octanol-water Partition Coefficients LogP of the protonic ionic liquids used in the electrochemical gas sensors prepared according to Examples 1 to 12 and Comparative Examples 1 to 16 are shown in Table 3 below.

The octanol-water partition coefficients LogP were calculated by using a commercial thermodynamic program COSMO-RS (version 2019.304, SCM). By using the program, surface charge distribution of each compound and chemical potential difference to water and octanol were calculated to obtain the partition coefficient LogP, wherein GGA: BP86_TZP parameter was applied thereto.

### Evaluation Example 3: Sensitivity

The electrochemical gas sensors (CO sensors) prepared according to Examples 1 to 12 and Comparative Examples 1 to 16 were exposed to 100 ppm of CO in the atmosphere. Sensitivities of the CO sensors were measured and the results are shown in Tables 3 and 4 below.

TABLE 3

| | | | Sensitivity | |
|---|---|---|---|---|
| Example | Cation | Anion | (nA/ppm cm²) | LogP |
| 1 | diEtNH₂ | HSO₄_{^{−}} | 32.2 | −3.7542 |
| 2 | triMeNH | HSO₄_{^{−}} | 37.56 | −4.3887 |
| 3 | PyrH | HSO₄_{^{−}} | 23.2 | −3.9664 |
| 4 | EtPyrH | HSO₄_{^{−}} | 18.19 | −3.8034 |
| 5 | MeOEtNHa | HSO₄_{^{−}} | 23.45 | −3.7575 |
| 6 | MeOEtNH₂Me | HSO₄_{^{−}} | 25.33 | −3.7482 |
| 7 | bis(MeOEt)NH₂ | HSO₄_{^{−}} | 22.11 | −3.5748 |
| 8 | EtPyrH | SO₃NH₂_{^{−}} | 5.94 | −4.2255 |
| 9 | diEtNH2 | SO₃NHMe | 10.01 | −3.6613 |
| 10 | EtPyrH | SO₃NHMe | 9.59 | −3.7104 |
| 11 | MeOEtNH₃ | SO₃NHMe | 8.53 | −3.6645 |
| 12 | MeOEtNH₂Me | SO₃NHMe | 10.29 | −3.6553 |

TABLE 4

| Comparative | | | | Sensitivity | |
|---|---|---|---|---|---|
| Example | Protonic ionic liquid | Cation | Anion | (nA/ppm cm²) | LogP |
| 1 | EMI-TFSI | EMI | TFSI | 0 | 1.32731621 |
| 2 | EMI-EtSO₄ | EMI | EtSO₄ | 1.26 | −3.0171 |
| 3 | Pyr13-TFSI | Pyr13 | TFSI | 0.15 | 1.4281 |
| 4 | Pyr14-OTf | Pyr14 | Otf | 0.3 | −1.30501 |
| 5 | Pyr14-DCN | Pyr14 | DCN | 0 | −2.01032 |
| 6 | EMI-DCN | EMI | DCN | 1.33 | −2.4294 |
| 7 | EMI-CCN | EMI | CCN | 0 | 1.3342 |
| 8 | EMI-SCN | EMI | SCN | 0.2 | −2.6447 |
| 9 | EMI-TFA | EMI | TFA | 0.3 | −1.9034 |
| 10 | EMI-diEtPO₄ | EMI | diEtPO₄ | 0.17 | −2.3058 |
| 11 | DEMHA-Otf | DEMHA | Otf | 0.43 | −1.9177 |
| 12 | BMI-HSO₄ | BMI | HSO₄_{^{−}} | 3.93 | −2.8365 |
| 13 | HEI-TFSI | HEI | TFSI | −0.22 | 1.3171 |
| 14 | HEI-NO₃ | HEI | NO₃ | 4.68 | −3.2114257 |
| 15 | EH3A-NO₃ | EH₃A | NO₃ | 3.18 | −3.4452894 |
| 16 | PH3A-NO₃ | PH₃A | NO₃ | 0.16 | −3.1646 |

The electrochemical gas sensors of Examples 1 to 12 use electrolytes including the protonic ionic liquid having a logP of −3.5 or less, and these electrochemical gas sensors have improved sensitivity compared to the electrochemical gas sensors of Comparative Examples 1 to 16 as shown in Tables 3 and 4.

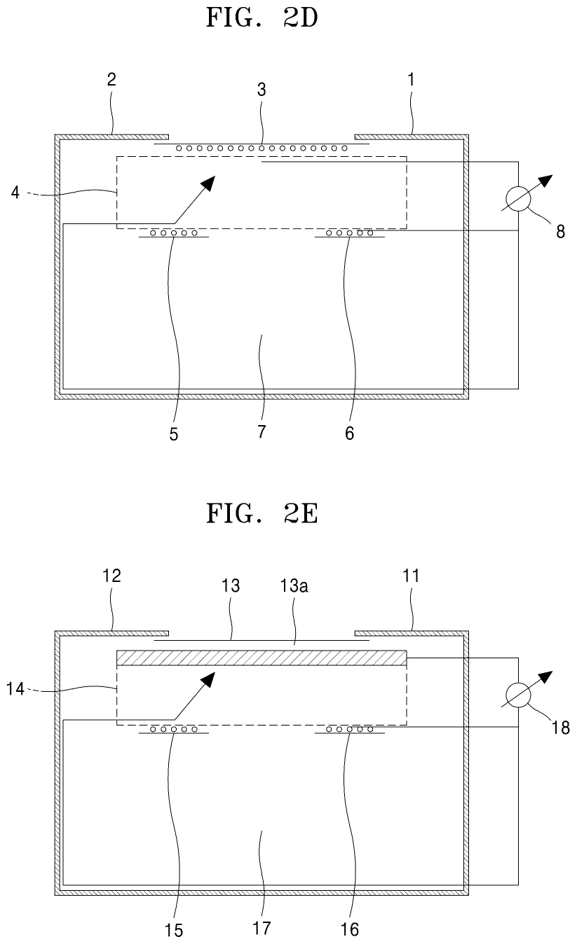

Sensitivities of the electrochemical gas sensors of the Examples and the Comparative Examples with respect to LogP were measured and shown in FIG. 3.

Referring to FIG. 3, it was confirmed that the sensitivity of the electrochemical gas sensor was significantly improved when the protonic ionic liquid having the LogP of −3.5 or less was used.

### Evaluation Example 4: Evaluation of Lifespan of Electrochemical Gas (O₂) Sensor

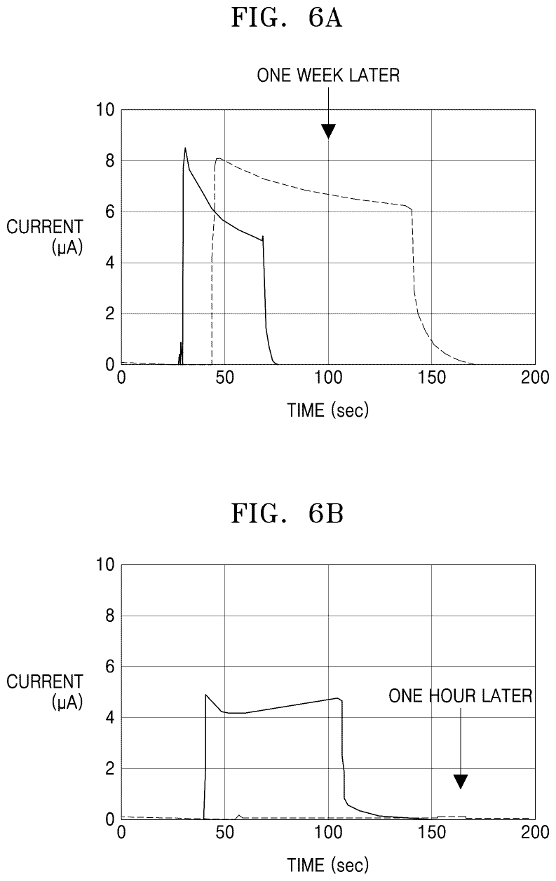

The electrochemical gas sensors of Example 13 and Comparative Example 17 were exposed to 100% O₂. A voltage and a current between the working electrode and the reference electrode of the electrochemical gas sensor were about −0.4 V and −8 μA, respectively. Sensor signals over time of the Example 13 and Comparative Example 17 are shown in graphs of FIGS. 6A and 6B.

While O₂ was not detected by the electrochemical gas sensor of Comparative Example 17 after 1 hour as shown in FIG. 6B, O₂ was able to be detected by the electrochemical gas sensor of the Example 1 after 1 week referring to FIG. 6A indicating a significant increase in useable time.

### Evaluation Example 5

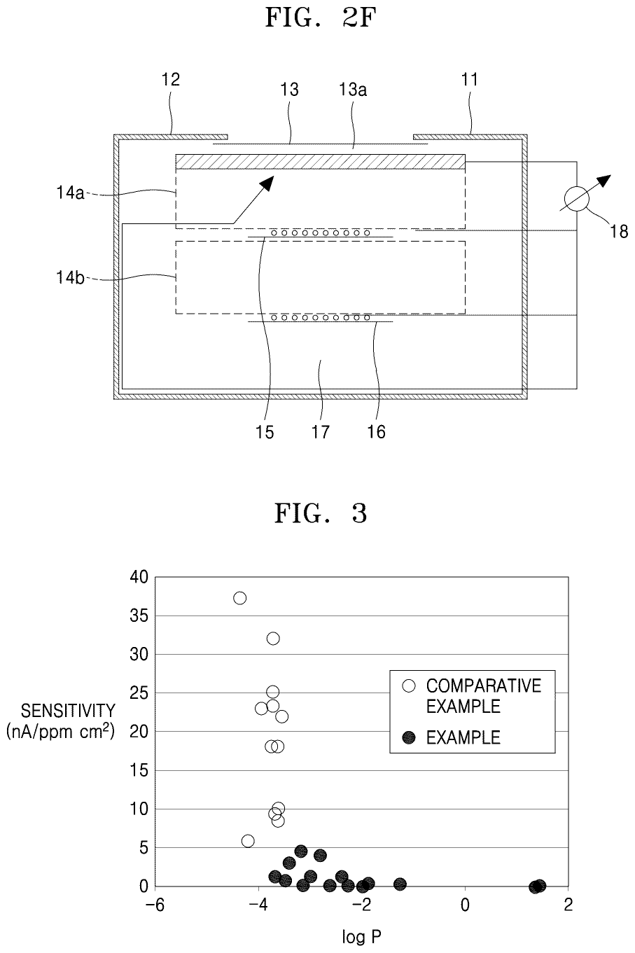

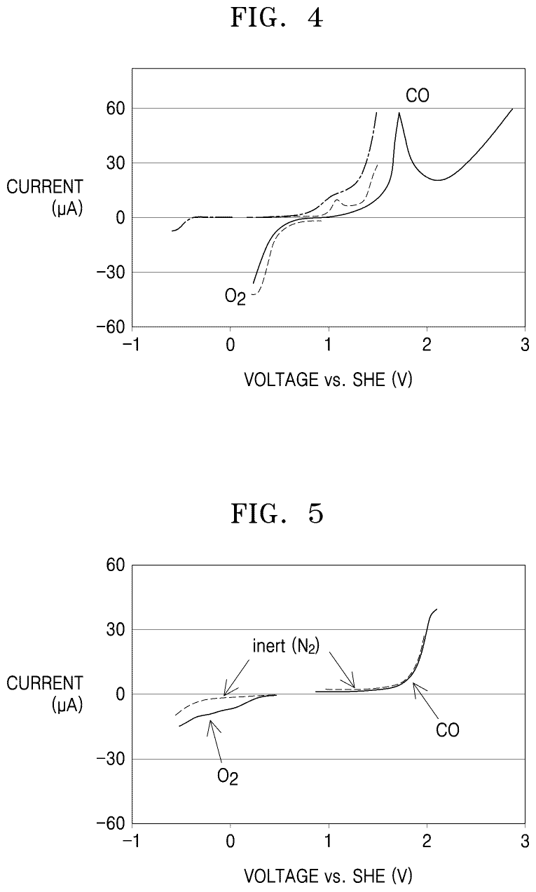

Currents of the protonic ionic liquids of Comparative Preparation Example 4, Preparation Example 1, Comparative Preparation Example 6 (when the pKa of an anion's conjugate acid was −14, −3, and 5) and the protonic ionic liquid of Comparative Preparation Example 1 (wherein the pKa of an anion's conjugate acid was −20), with respect to voltage, were measured and are shown in FIGS. 4 and 5, respectively.

When the protonic ionic liquid having the pKa of the anion's conjugate acid of −14, −3, or 5 is used, CO and O₂ may be detected as shown in FIG. 4. However, when the protonic ionic liquid having the pKa of the anion's conjugate acid of −20 is used, it is difficult to detect O₂ and CO as shown in FIG. 5.

### Evaluation Example 6: Viscosity

Viscosity of the protonic ionic liquid of Preparation Example 10 was measured and the results are shown in FIG. 5.

TABLE 5

| Preparation Example | Viscosity (cP) |
|---|---|
| Preparation Example 10 | 29 |

Because the protonic ionic liquid of Preparation Example 10 has a low viscosity lower than 100 cP, mobility of the protonic ionic liquid may be increased.

Because the electrolyte for electrochemical gas sensors according to one or more embodiments has nonvolatility and hydrophilicity, a miniaturized electrochemical gas sensor may be manufactured. Such an electrochemical gas sensor may have improved sensitivity and may be used with a small power.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An electrolyte for an electrochemical gas sensor, the electrolyte comprising:
a protonic ionic liquid,
wherein the protonic ionic liquid has an octanol-water partition coefficient LogP of about −3.5 or less,
wherein water is used as a reactant of an electrochemical reaction for gas sensing, the water is generated as a product of the electrochemical reaction for gas sensing, or a combination thereof,
wherein the protonic ionic liquid is a compound represented by Formula 1, wherein two of R¹ to R³ are linked to together to form a ring, wherein the ring is represented by a ring of Formula 1-1: 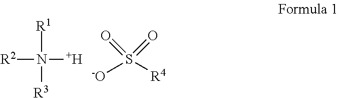
wherein in the Formula 1,
R⁴ is OH, NH₂, NH (R⁵), or N (R⁵) (R⁶), and R⁵ and R⁶ are each independently a substituted or unsubstituted C1-C10 alkyl group, a substituted or unsubstituted C6-C20 aryl group, a substituted or unsubstituted C7-C20 alkylaryl group, or a substituted or unsubstituted C7-C20 arylalkyl group, 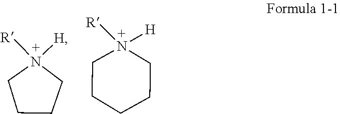
wherein in Formula 1-1, R′ is a substituted or unsubstituted C1-C5 alkyl group.

2. The electrolyte of claim 1,
wherein the octanol-water partition coefficient of the protonic ionic liquid is from about −4.5 to about −3.5.

3. The electrolyte of claim 1,
wherein the protonic ionic liquid comprises an anion, and
wherein a pKa of a conjugate acid of the anion is about −14 or greater.

4. The electrolyte of claim 1,
wherein a vapor pressure of the protonic ionic liquid is about 0.1 pascals or less at 25° C., and
a a viscosity of the protonic ionic liquid is about 100 centipoise or less.

5. The electrolyte of claim 1,
wherein an ionic conductivity of the protonic ionic liquid is 1×10⁻⁷ Siemens per centimeter or greater.

6. The electrolyte of claim 1, wherein the protonic ionic liquid comprises:
a protonic cation having a water binding energy of about 0.6 kilocalories per mole or less, and
an anion having a water binding energy of about 7 kilocalories per mole or less.

7. The electrolyte of claim 1,
wherein the ring is represented by a ring of Formula 1-2:

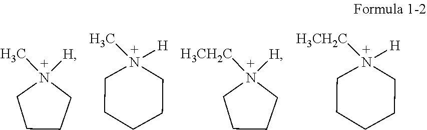

8. The electrolyte of claim 1,
wherein the R⁴ is —NH₂, —NHCH₃, or —NCH₂CH₃.

9. The electrolyte of claim 1,
wherein the protonic ionic liquid comprises a compound represented by at least one of Formulae 6 to 11, or 19:

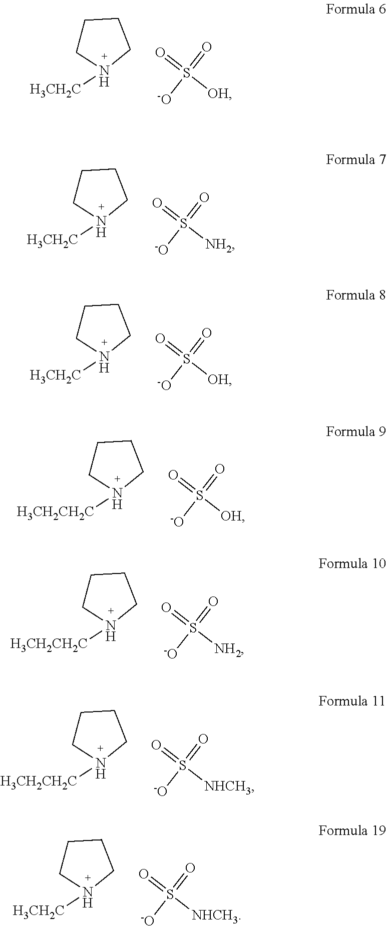

10. The electrolyte of claim 1,
wherein, in the electrochemical reaction for gas sensing in the electrochemical gas sensor, the protonic ionic liquid detects at least one of CO, H₂S, SO₂, NO, NO₂, O₂, O₃, or a combination thereof.

11. The electrolyte of claim 1,
wherein a content of the protonic ionic liquid in the electrolyte is from about 30 parts by weight to about 100 parts by weight based on 100 parts by weight of a total weight of the electrolyte.

12. An electrochemical gas sensor, comprising the electrolyte according to claim 11.

13. The electrochemical gas sensor of claim 12,
wherein the electrochemical gas sensor comprises two or more electrodes and the electrolyte comprises the protonic ionic liquid ionically in contact with the two or more electrodes.

14. The electrochemical gas sensor of claim 12,
wherein the electrochemical gas sensor operates at a voltage of about 1 volt or greater.

15. The electrochemical gas sensor of claim 13,
wherein the two or more electrodes comprise at least one of platinum (Pt), gold (Au), palladium (Pd), iridium (Ir), silver (Ag), ruthenium (Ru), nickel (Ni), stainless steel, aluminum (AI), molybdenum (Mo), chromium (Cr), copper (Cu), titanium (Ti), tungsten (W), tin-doped In203, fluorine-doped SnO₂, rhodium (Rh), an oxide of at least one metal, carbon, or a combination thereof.

16. The electrochemical gas sensor of claim 13,
wherein the two or more electrodes comprise a working electrode, a counter electrode, and a reference electrode, and
wherein the electrochemical gas sensor further comprises:
a first separator interposed between the working electrode and the reference electrode,
a a second separator interposed between the reference electrode and the counter electrode, and
a a moisturizing member.

17. The electrochemical gas sensor of claim 12,
wherein the electrochemical gas sensor comprises:
an insulating substrate;
two or more electrodes located on at least one surface of the insulating substrate; and
the protonic ionic liquid located on the two or more electrodes.

18. The electrochemical gas sensor of claim 12,
wherein the electrochemical gas sensor further comprises a porous gas-permeable membrane.

19. The electrochemical gas sensor of claim 12,
wherein the electrolyte comprises about 70 parts by weight or less of water based on 100 parts by weight of a total weight of the electrolyte, and
an amount of water evaporation after 5 minutes is about 0.01 weight percent or less when a temperature of the electrochemical gas sensor is 25° C.

20. The electrochemical gas sensor of claim 12,
wherein a sensitivity of the electrochemical gas sensor is from about 5 nanoamperes per part per million square centimeters to 50 nanoamperes per part per million square centimeters.

21. The electrochemical gas sensor of claim 12,
wherein the electrochemical gas sensor is a CO gas sensor.

22. The electrochemical gas sensor of claim 12,
wherein a usage time of the electrochemical gas sensor is from about 1 week to about 5 years.

23. A protonic ionic liquid, represented by Formula 2: 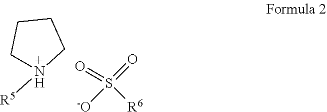
wherein in Formula 2,
R⁵ is a substituted or unsubstituted C2-C10 alkyl group,
R⁶ is OH, —NH₂, or —NHR, and
R is a C1-C10 substituted or unsubstituted alkyl group or a substituted or unsubstituted C6-C20 aryl group.

24. The protonic ionic liquid of claim 23,
wherein the protonic ionic liquid is a compound represented by one of Formulae 3 to 5: 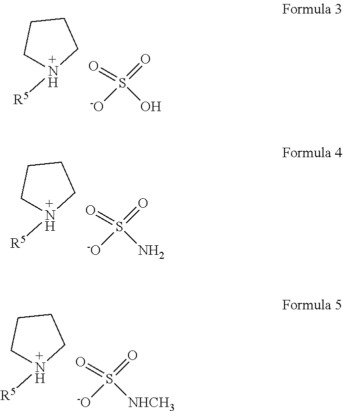
wherein in Formulae 3 to 5,
R⁵ is a substituted or unsubstituted C2-C10 alkyl group.

25. The protonic ionic liquid of claim 23,
wherein the protonic ionic liquid is a compound represented by one of Formulae 6 to 11:

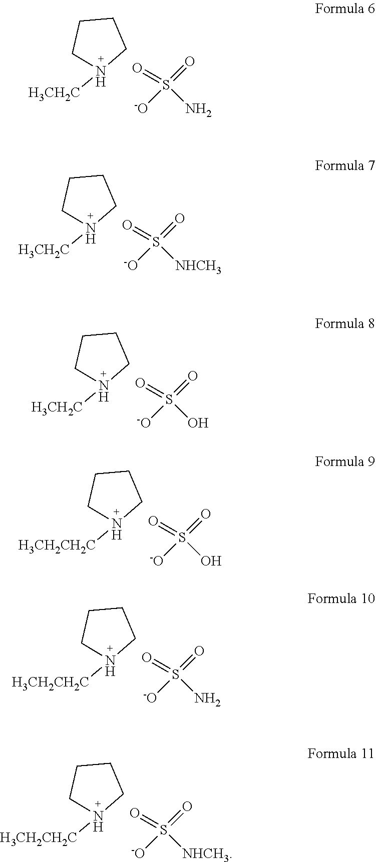

26. The protonic ionic liquid of claim 23,
wherein an octanol-water partition coefficient LogP of the protonic ionic liquid is about −3.5 or less.

27. An electrochemical device, comprising an electrolyte comprising the protonic ionic liquid according to claim 23.

28. The electrochemical device of claim 27,
wherein the electrochemical device is a gas sensor, a battery, a biosensor, or a fuel cell.

29. The electrochemical device of claim 27,
wherein the electrochemical device is an electrochemical gas sensor, and an octanol-water partition coefficient LogP of the electrolyte is about −3.5 or less.
